SEQUENCE LISTING (54 sequences)

SEQ ID NO: 1 - DNA, 503 bp, human
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 2 - DNA, 1513 bp, human
cgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaaaaaaaaaaaaaaaaaaaaaaaaaa

SEQ ID NO: 3 - DNA, 620 bp, human
ggggacttcttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaatcggttttatactattgaaatcctaaaggtagaataatggaagccctgtctgtttgccacacccaggtgatttcctctaaagaaacttggctggaatttctgctgtggtctataaaataaacttcttaacatgcttctcctgaaaaaaaaaaaaaaaaaaaaaaaaaaaaaa

SEQ ID NO: 4 - DNA, 1155 bp, human
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcagcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagcaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaattaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 5 - DNA, 3865 bp, human
tccgagctgattacagacaccaaggaagatgctgtaaagagtcagcagccacagccctggctagctggccctgtgggcatttattagtaaagttttaatgacaaaagctttgagtcaacacacccgtgggtaattaacctggtcatccccaccctggagagccatcctgcccatgggtgatcaaagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaagagtctcctaataaagatggtcttctgaaggctacctgcggaatgaaagtttctattccaactaaagccttagaattgaaggacatgcaaactttcaaagcagagcctccggggaagccatctgccttcgagcctgccactgaaatgcaaaagtctgtcccaaataaagccttggaattgaaaaatgaacaaacattgagagcagatgagatactcccatcagaatccaaacaaaaggactatgaagaaagttcttgggattctgagagtctctgtgagactgtttcacagaaggatgtgtgtttacccaaggctrcrcatcaaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctrcrcatcaaaaagaaatagataaaataaatggaaaattagaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaasgttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgcmggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaaccagaatctcgctctgtcactcaggctgg

SEQ ID NO: 6 - DNA, 3282 bp, human
gggacagggctgaggatgaggagaaccctggggacccagaagaccgtgccttgcccggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttgtaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaagcattgtttttatataaaaacaatgataaagatgtgaactgtgaaataaatataccatattagctacccaccaaaaaaaaaaaaaaaaaa

SEQ ID NO: 7 - DNA, 1894 bp, human
gtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactg

SEQ ID NO: 8 - DNA, 1377 bp, human
cacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctagcaacagctggcctgcagcgcatgggctggcacaaccgggtggggcagatcctgcaccctgagaaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggt

SEQ ID NO: 9 - DNA, 21 bp, human
agttgctgatggtcctcatgc

SEQ ID NO: 10 - DNA, 23 bp, human
atcacattctccaataaggggca

SEQ ID NO: 11 - DNA, 25 bp, human
ccctctcccagcactgctacgcatt

SEQ ID NO: 12 - DNA, 22 bp, human
cacccttcagggtcttgagatt

SEQ ID NO: 13 - DNA, 20 bp, human
tccgtttctgccagtgtgtc

SEQ ID NO: 14 - DNA, 26 bp, human
acagctgagcatgaaagctgcctttt

SEQ ID NO: 15 - DNA, 22 bp, human
gcctactttccaagcggagcca

SEQ ID NO: 16 - DNA, 19 bp, human
ttgcgggtacccacgcgaa

SEQ ID NO: 17 - DNA, 27 bp, human
aacggcaatgcggctgcaacggcggaa

SEQ ID NO: 18 - DNA, 29 bp, human
caaacggatgaaactctgagcaatgttga

SEQ ID NO: 19 - DNA, 24 bp, human
tctgtgagccaaaggtcttgcaga

SEQ ID NO: 20 - DNA, 36 bp, human
tgtttatgcaattaatatatgacagcagtctttgtg

SEQ ID NO: 21 - DNA, 21 bp, human
agatgagcaggtccgaggtta

SEQ ID NO: 22 - DNA, 23 bp, human
cctgattctgccgctcactatca

SEQ ID NO: 23 - DNA, 29 bp, human
acccttcagggtcttgagattgagctgca

SEQ ID NO: 24 - DNA, 21 bp, human
gcaagtgccaatgatcagagg

SEQ ID NO: 25 - DNA, 50 bp, human
atatagactcaggtatacacacttcccatcagaatccaaacaagaggaag

SEQ ID NO: 26 - DNA, 21 bp, human
tctgataaaggccgtacaatg

SEQ ID NO: 27 - DNA, 22 bp, human
tcacgacttgctgtttttgctc

SEQ ID NO: 28 - DNA, 27 bp, human
atcaaaaaacaagcatggcctcacacc

SEQ ID NO: 29 - DNA, 21 bp, human
gcttggtggttaaaacttacc

SEQ ID NO: 30 - DNA, 20 bp, human
tgaacagttctgttggtgta

SEQ ID NO: 31 - DNA, 27 bp, human
ctgcctgcctatgtgacgacaatccgg

SEQ ID NO: 32 - DNA, 22 bp, human
gccgcttcattaggtggctcaa

SEQ ID NO: 33 - DNA, 22 bp, human
agcggctcagcttgtcgtagtt

SEQ ID NO: 34 - DNA, 35 bp, human
aaggagaagggcatcttcaaaattgaggactcagc

SEQ ID NO: 35 - DNA, 20 bp, human
caattttggtggagaacccg

SEQ ID NO: 36 - DNA, 20 bp, human
gctgtcggaggtatatggtg

SEQ ID NO: 37 - DNA, 28 bp, human
catttcagagagtaacatggactacaca

SEQ ID NO: 38 - DNA, 22 bp, human
ctgcttcgctgcatcgctgaaa

SEQ ID NO: 39 - DNA, 22 bp, human
cagactcctccagtcaggtaca

SEQ ID NO: 40 - DNA, 30 bp, human
cctgaggcacctggaaggaggctgcagtgt

SEQ ID NO: 41 - DNA, 25 bp, human
cggatgaaactctgagcaatgttga

SEQ ID NO: 42 - DNA, 23 bp, human
gagccaaaggtcttgcagaaagt

SEQ ID NO: 43 - DNA, 36 bp, human; identical to SEQ ID NO: 20

SEQ ID NO: 44 - DNA, 22 bp, human
gcactgctacgcaggctctggc

SEQ ID NO: 45 - DNA, 21 bp, human
gcagctttcatgctcagctgt

SEQ ID NO: 46 - DNA, 20 bp, human
cacccttcagggtcttgaga

SEQ ID NO: 47 - DNA, 20 bp, human
tccgtttctgccagtgtgtc

SEQ ID NO: 48 - DNA, 24 bp, human
gctgagcatgaaagctgccttgga

SEQ ID NO: 49 - DNA, 21 bp, human
cacccttcagggtcttgagat

SEQ ID NO: 50 - DNA, 22 bp, human
acccttcagggtcttgagattg

SEQ ID NO: 51 - DNA, 23 bp, human
acccttcagggtcttgagattga

SEQ ID NO: 52 - DNA, 20 bp, human
ctccgtttctgccagtgtgt

SEQ ID NO: 53 - DNA, 20 bp, human
acccacgcgaatcactctca

SEQ ID NO: 54 - DNA, 19 bp, human
caagcggagccatgtctgg

(19) United States
(12) Patent Application Publication
(10) Pub. No.: US 2009/0298052 A1
(43) Pub. Date: Dec. 3, 2009

(54) Diagnosing or Predicting the Course of Breast Cancer

(76) Inventors: David Atkins, Bucks (GB); John Backus, Rochester, NJ (US); Robert Belly, Webster, NJ (US); Steven Rosen, Mountain Lakes, NJ (US); Robert White, Rochester, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/569,992

(22) PCT Filed: Jun. 6, 2005
(86) PCT No.: PCT/US05/19616

§ 371 Date: Jun. 5, 2009

Related U.S. Application Data
(60) Provisional application No. 60/577,155, filed on Jun. 4, 2004.

(51) Int. Cl. C12Q 1/68

(52) U.S. Cl. 435/6

(57) ABSTRACT

A method of diagnosing the presence or predicting the course of breast cancer by measuring the expression of a combination of Marker genes comprising a tissue-specific gene and a non-tissue specific gene in a cell or tissue sample derived from a patient. In one aspect of the invention, the genes are mammaglobin and CK19. Kits, nucleic acid primers and probes and controls are provided.

### FIELD OF THE INVENTION

The invention relates to the field of molecular diagnostics particularly in breast cancer.

### BACKGROUND

Lymph node involvement is the strongest prognostic factor in many solid tumors, and detection of lymph node micrometastases is of great interest to pathologists and surgeons. Current lymph node evaluation involves microscopic examination of H&E-stained tissue sections and suffers from three major limitations: (a) single tumor cells, or small foci of cells, are easily missed; (b) the result is not rapidly available, meaning that any positive result in a sentinel lymph node (SLN) procedure requires a second surgery for removal of axcillary lymph nodes and (c) only one or two tissue sections are studied, and thus the vast majority of each node is left unexamined. Serial sectioning can help overcome the issue of sampling error, and immunohistochemistry (IHC) can help identify individual tumor cells. The combination of these methods, however, is too costly and time consuming for routine analysis and is limited to special cases such as SLN examination.

Surgical decisions are often based on intra-operative frozen section analysis of lymph nodes; however, the sensitivity of these methods is relatively poor, ranging from 50-70% relative to standard H&E pathology, leading to an unacceptably high rate of second surgeries. The five-year survival of Stage 0 and I breast cancer patients who do not have lymph node involvement are 92% and 87%, respectively. On the other hand, the five-year survival of later stage breast cancer patients who do have lymph node involvement decrease significantly. For example, the survival of Stage II breast cancer is only 75%, Stage III 46%, and Stage IV 13%. Although node negative breast cancer patients have improved survival, 20-30% of histologically node negative patients suffer disease recurrence. This is most likely due to the limitations of current techniques in the detection of micrometastases including issues related to node sampling and poor sensitivity for detecting individual tumor cells or small tumor foci.

In addition to a need for more accurate and sensitive detection of metastases in breast cancer nodes, there is a need for more accurate detection of surgical margins, particularly in the intraoperative setting. The polymerase chain reaction (PCR) is a powerful tool in the field of molecular biology that could be useful in this setting. This technique allows for replicating/amplifying small amounts of nucleic acid fragments into quantities that can be analyzed in a meaningful way. Furthermore with the development of real-time quantitative RT-PCR (Q-RT-PCR), this technology has become more reliable as well as amenable to automation. Q-RT-PCR is less subject to contamination and provides quantitation of gene expression. Such quantitation could be applied for the detection of micrometastases in intraoperative lymph node assays. PCR in molecular diagnostics, despite its advantages, has several limitations that make it difficult to apply in typical clinical diagnostic setting, particularly in the intraoperative setting.

One such limitation is the time it typically takes to perform PCR diagnoses. Typical PCR reactions take hours, not minutes. Decreasing the time it takes to carry out a PCR reaction is necessary if the technique is to be useful intraoperatively. Further, although Q-RT-PCR can provide quantitative results, to date there have been no known cutoff values for distinguishing positive from negative results based on such technology nor has it been clear which nucleic acid fragments are best detected and correlated to the presence of a micrometastasis. Other methods for the amplification and detection of nucleic acid fragments exist as well and each suffers from similar problems.

An intraoperative molecular lymph node assay that overcomes the existing difficulties would be well accepted by the medical community.

Cytokeratin 19 (also known as Keratin 19, CK19 and KRT19) has been recognized as a useful gene for the detection of epithelial cells. The detection of these cells in several body compartments (including blood, bone marrow and lymph nodes) is associated with metastasis of several cancers, including breast cancer. Detection of CK19 mRNA is often complicated by the presence of four pseudogenes (one on Chromosome 6, one on Chromosome 4 and two on Chromosome 12). The sequences of these pseudogenes do not have intronic regions that allow for discrimination between spliced mRNA and DNA and have up to 90% homology with the entire CK19 mRNA sequence. Designing primers and probes that discriminate between CK19 mRNA and CK19 DNA and also discriminate CK19 mRNA from the four pseudogenes has proven to be a challenge. While primers have been published that discriminate between CK19 mRNA from DNA, other groups have found these primers to cross-react with DNA. To avoid the issue of cross-reactivity with DNA, many groups employ RNA purification methods that either: (1) include a DNA degradation step (with DNase) or (2) are based on methodologies, such as Trizol, that remove >99% of contaminating DNA.

It is typically undesirable to require a DNA degradation step or to require that Trizol-based RNA purification is employed. Both methods increase the time and complexity required for RNA preparation. Thus, these methods are not suited for applications that require a combination of high ease-of-use and rapidity, such as is the case with intra-operative applications. In cases such as these, is it necessary to employ CK19 amplification methods that can discriminate between mRNA and DNA.

### SUMMARY OF THE INVENTION

The invention is an assay for diagnosing the presence of or predicting the course of breast cancer. In one embodiment, the assay diagnoses micrometastases. In another embodiment, detection of micrometastases is in an SLN, particularly during surgery. A surgeon identifies a SLN during surgery according to known methods. SLNs are removed and prepared as described below. Nucleic acid (e.g., DNA and RNA) is then rapidly extracted from the SLNs. Markers indicative of micrometastases, if present, are then amplified and detected. The surgeon then takes action based upon the outcome of the detection of such Markers.

In another aspect of the invention, the Markers are nucleic acid fragments specific for a particular tissue and at least one Marker that is not tissue specific.

In yet another aspect of the invention, the Markers are nucleic acid fragments indicative of malignancy.

In yet another aspect of the invention the Markers are those of mammaglobin (SEQ ID NO:1 and CK19 (SEQ ID NO: 2) or either PIP (SEQ ID NO: 3), B305D (particularly, isoform C, SEQ ID NO: 4), B726 (SEQ ID NO: 5), GABA (SEQ ID NO:6) or PDEF (SEQ ID NO: 7) in the case of breast cancer diagnostics. See, respectively, Watson et al. (1996) Cancer Res. 56:860-865, Hoffman-Fazel et al. (2003) Anticancer Res. 23:917-920, Strausberg et al. (2002) Proc. Natl. Acad. Sci. USA 99:16899-16903, Zehentner et al. (2002) Clin. Chem. 48:1225-1231 (B305D and B726), Mehta et al. (1999) Brain Res. Brain Res. Rev. 29:196-217, Feldman et al. (2003) Cancer Res. 63:4626-4631, and Grandchamp et al. (1987) Eur. J. Biochem. 162:105-110.

The present invention defines specific primer/probe sets that optimally amplify and mammaglobin RNA and detect the amplification products.

In another aspect of the invention, optimal primers and probes are disclosed for the specific detection of CK19 mRNA.

In yet another aspect of the invention, micrometastases are detected by a method that includes the steps of: obtaining RNA from an SLN; performing a quantitative RT-PCR method specific to two or more genes of interest and determining if the presence of the Markers exceed a predetermined cut-off. The cut-off values can be an absolute value or a value relative to the expression of a control gene.

In another aspect of the invention, the assays include DNA encoding both a constitutively expressed internal control gene and the Markers for use in providing controls for reaction quality and adequacy of all RNA-related portions of the assay. In one aspect, the internal control gene is porphobilinogen deaminase (PBGD, SEQ ID NO: 8).

In a yet further embodiment of the invention, kits contain reagents for conducting the assays.

### DESCRIPTION OF THE DRAWING

FIG. 1 is a bar graph depicting sensitivity of individual Markers at 95% specificity.

### DETAILED DESCRIPTION

Methods for cancer diagnostics and predictions are presented. These methods employ extracting nucleic acids from cells or a tissue such as a lymph node and a method of amplifying and detecting nucleic acid fragments indicative of breast cancer (such fragments are referred to herein as “Markers”).

If the assays are to be performed intraoperatively, the rapid amplification and detection of Markers indicative of the expression of certain genes is essential. Provided that such methods can be conducted within a period acceptable for an intraoperative assay (i.e., no more than about 35 minutes), any reliable, sensitive, and specific method can be used. This includes PCR methods, Rolling Circle Amplification methods (RCA), Ligase Chain Reaction methods (LCR), Strand Displacement Amplification methods (SDA), Nucleic Acid Sequence Based Amplification methods (NASBA), and others. The rapid molecular diagnostics involved are most preferably quantitative PCR methods, including QRT-PCR.

Irrespective of the amplification method employed, it is important to adequately sample the tissue used to conduct the assay. In the case of SLNs, this includes proper excision and processing of the SLN as well as extraction of RNA from it. Once obtained, it is important to process the nodes properly so that any cancerous cells present are detected.

A variety of techniques are available for extracting nucleic acids from tissue samples. Standard practice in each case is time consuming and can be difficult even when using a commercially available kit designed for this purpose. Typical commercially available nucleic acid extraction kits take at least 15 minutes to extract the nucleic acid. In the methods of the instant invention, nucleic acid is extracted in less than 8 minutes and preferably less than 6 minutes. These rapid extraction methods are the subject of U.S. patent application Ser. No. 10/427,217.

The successful isolation of intact RNA generally involves four steps: effective disruption of cells or tissue, denaturation of nucleoprotein complexes, inactivation of endogenous ribonuclease (RNAase) and removal of contaminating DNA and protein. The disruptive and protective properties of guanidinium thiocyanate (GTC) and B-mercaptoethanol to inactivate the ribonucleases present in cell extracts make them preferred reagents for the first step. When used in conjunction with a surfactant such as sodium dodecylsulfate (SDS), disruption of nucleoprotein complexes is achieved allowing the RNA to be released into solution and isolated free of protein. Dilution of cell extracts in the presence of high concentrations of GTC causes selective precipitation of cellular proteins to occur while RNA remains in solution. Centrifugation can clear the lysate of precipitated proteins and cellular DNA and is preferably performed through a column. Such columns also shear DNA and reduce the viscosity of the sample. RNA purification is preferably conducted on a spin column containing silica or other material. Manual cell and tissue disruption can be by means of a disposable tissue grinder as described in U.S. Pat. No. 4,715,545. Homogenization time is within 1 to 2 minute and is more preferably 30-45 sec.

The sample can then processed with a shredding column (e.g., QIAshredder, QIAGEN Inc., Valencia, Calif., or suitable substitute) or with an RNA processing device such as the PCR Tissue Homogenization Kit commercially available from Omni International (Warrenton, Va.) to reduce its viscosity. RNA is precipitated out via the spin column as described above and centrifugation times are no greater than 30 sec. When using commercial RNA extraction kits such as those available from Qiagen, Inc., filtration is used instead of centrifugation for all steps except for the column drying and RNA elution steps. Typically, the sample is diluted with an equal volume of 70% ethanol prior to application on the column. After washes by filtration, the column is dried by centrifugation, and RNA is eluted in RNAase free water. The RNA is selectively precipitated out of solution with ethanol and bound to a substrate (preferably, a silica-containing membrane or filter). The binding of RNA to the substrate occurs rapidly due to the disruption of the water molecules by the chaotropic salts, thus favoring absorption of nucleic acids to the silica. The bound total RNA is further purified from contaminating salts, proteins and cellular impurities by simple washing steps. Finally, the total RNA is eluted from the membrane by the addition of nuclease-free water. The total time of this rapid protocol is less than 8 minutes and preferably less than 6 min.

In summary the rapid RNA extraction method involves the following steps:

(a) obtaining a sample containing cells from the biological system,
(b) optionally, removing from the sample, cells without RNA of interest to produce a working sample,
(c) lysing the cells containing RNA that is of interest and producing a homogenate of them,
(d) optionally, diluting the homogenate,
(e) contacting the wetted, homogenized working sample with a substrate containing, or to which is affixed, a material to which RNA binds,
(f) allowing the sample to bind to the substrate,
(g) removing contaminants and interferents,
(h) drying the substrate, and
(i) eluting RNA from the substrate;
in instances in which centrifugation is used, it may occur after steps g, h, or I and vacuum/filtration is preferably applied in extraction steps.
The reagents involved in this rapid extraction process are:
Lysis/Binding buffer (preferably, 4.5M guanidinium-HCl, 100 mM NaPO₄),
Wash buffer I (preferably, 37% ethanol in 5M guanidine-HCl, 20 mM Tris-HCl),
Wash buffer II (preferably, 80% ethanol in 20 mM NaCl, 2 mM Tris-HCl), Elution buffer, and
Nuclease-free sterile double distilled water.

Since the distribution of cancer cells in nodes is non-uniform, it is preferable that multiple sections of the node be sampled. Optionally, one or more nodes may also be examined based on pathology. One method for accomplishing both a molecular based test and an examination of the same node sample by pathology is to section the node into at least four sections with one outer and inner section used for pathology, and one outer and inner section for used for molecular testing. As the distribution of metastases and micrometastases in tissues is not uniform in nodes or other tissues, a sufficiently large sample should be obtained so that metastases will not be missed. One approach to this sampling issue in the present method is to homogenize a large tissue sample, and subsequently perform a dilution of the well-mixed homogenized sample to be used in subsequent molecular testing.

A typical PCR reaction includes multiple amplification steps, or cycles that selectively amplify target nucleic acid species. A typical PCR reaction includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 20 or more cycles of denaturation, annealing and elongation. In many cases, the annealing and elongation steps can be performed concurrently, in which case the cycle contains only two steps.

In the inventive method, employing RT-PCR, the RT-PCR amplification reaction is conducted in a time suitable for intraoperative diagnosis, the lengths of each of these steps can be in the seconds range, rather than minutes. Specifically, with certain new thermal cyclers being capable of generating a thermal ramp rate of at least about 5° C. per second, RT-PCR amplifications in 30 minutes or less are used. More preferably, amplifications are conducted in less than 25 minutes. With this in mind, the following times provided for each step of the PCR cycle does not include ramp times. The denaturation step may be conducted for times of 10 seconds or less. In fact, some thermal cyclers have settings for “0 seconds” which may be the optimal duration of the denaturation step. That is, it is enough that the thermal cycler reaches the denaturation temperature. The annealing and elongation steps are most preferably less than 10 seconds each, and when conducted at the same temperature, the combination annealing/elongation step may be less than 10 seconds. Some homogeneous probe detection methods, however, may require a separate step for elongation to maximize rapid assay performance. In order to minimize both the total amplification time and the formation of non-specific side reactions, annealing temperatures are typically above 50° C. More preferably annealing temperatures are above 55° C.

A single combined reaction for RT-PCR, with no experimenter intervention, is desirable for several reasons: (1) decreased risk of experimenter error, (2) decreased risk of target or product contamination and (3) increased assay speed. The reaction can consist of either one or two polymerases. In the case of two polymerases, one of these enzymes is typically an RNA-based DNA polymerase (reverse transcriptase) and one is a thermostable DNA-based DNA polymerase. To maximize assay performance, it is preferable to employ a form of “hot start” technology for both of these enzymatic functions. U.S. Pat. Nos. 5,411,876 and 5,985,619 provide examples of different “hot start” approaches. Preferred methods include the use of one or more thermoactivation methods that sequester one or more of the components required for efficient DNA polymerization. U.S. Pat. Nos. 5,550,044 and 5,413,924 describe methods for preparing reagents for use in such methods. U.S. Pat. No. 6,403,341 describes a sequestering approach that involves chemical alteration of one of the PCR reagent components. In the most preferred embodiment, both RNA- and DNA-dependent polymerase activities reside in a single enzyme. Other components that are required for efficient amplification include nucleoside triphosphates, divalent salts and buffer components. In some instance, non-specific nucleic acid and enzyme stabilizers may be beneficial.

The specificity of any given amplification-based molecular diagnostic relies heavily, but not exclusively, on the identity of the primer sets. The primer sets are pairs of forward and reverse oligonucleotide primers that anneal to a target DNA sequence to permit amplification of the target sequence, thereby producing a target sequence-specific amplicon. The primers must be capable of amplifying Markers of the disease state of interest. In the case of the instant invention, these Markers are directed to breast cancer.

In the case of breast cancer, the inventive method involves the amplification of a tissue marker specific for either breast tissue or breast cancer tissue and amplification a non-tissue specific Marker. The non-tissue specific Marker is preferably epithelial cell-specific. Suitable epithelial cell-specific Markers include, without limitation, lumican, selenoprotein P, connective tissue growth factor, keratin 19 (CK19), EPCAM, E-cadherin, and collagen, type IV, α-2. Combinations of at least two Markers are used such that clinically significant and reliable detection of breast/and or cancer cells in lymph nodes is detected when present. Preferably, the Markers are amplified and detected in a single reaction vessel at the same time (i.e., they are multiplexed). Most preferably, the primer/probe sets are complementary to nucleic acid fragments specific to those Markers.

The Markers include mammaglobin (SEQ ID NO: 1) and Cytokeratin 19 (CK19, SEQ ID NO: 2) or (preferably one) of the following in place of, or in addition to, mammaglobin: B305D (SEQ ID NO: 4), prolactin induced protein (PIP, SEQ ID NO: 3), B726 (SEQ ID NO: 5), GABA-π (SEQ ID NO: 6) or prostate derived Ets-transcription factor (PDEF, SEQ ID NO: 7). The combination of a tissue specific marker and a cancer specific marker provide sensitivity and specificity that exceeds 90% and 95% respectively. Surprisingly, the combination of a non-tissue specific Marker (CK19, SEQ ID NO: 2) and a cancer specific Marker (mammaglobin, SEQ ID NO: 1) provides even higher sensitivity and specificity, 91% and 97%, respectively. Some Markers exist in various isoforms with certain of the isoforms being more specific for one tissue or cancer than others. In the case of B305D, the most preferred isoform is B305D isoform C (SEQ ID NO: 4). It is also the most preferred Marker in combination with the mammaglobin and CK19 Markers.

The reaction must also contain some means of detection of a specific signal. This is preferably accomplished through the use of a reagent that detects a region of DNA sequence derived from polymerization of the target sequence of interest. Preferred reagents for detection give a measurable signal differential when bound to a specific nucleic acid sequence of interest. Often, these methods involve nucleic acid probes that give increased fluorescence when bound to the sequence of interest. The progress of the PCR reactions of the inventive method are typically monitored by analyzing the relative rates of amplicon production for each PCR primer set. Monitoring amplicons production may be achieved by a number of detection reagents and methods, including without limitation, fluorescent primers, fluorogenic probes and fluorescent dyes that bind double-stranded DNA, molecular beacons, Scorpions, and others.

A common method of monitoring a PCR reaction employs a fluorescent hydrolysis probe assay exploiting the 5′ nuclease activity of certain thermostable DNA polymerases (such as Taq or Tfl DNA polymerases) to cleave an oligomeric probe during the PCR process. The oligomer is selected to anneal to the amplified target sequence under elongation conditions. The probe typically has a fluorescent reporter on its 5′ end and a fluorescent quencher of the reporter at the 3′ end. So long as the oligomer is intact, the fluorescent signal from the reporter is quenched. However, when the oligomer is digested during the elongation process, the fluorescent reporter is no longer in proximity to the quencher. The relative accumulation of free fluorescent reporter for a given amplicon may be compared to the accumulation of the same amplicons for a control sample and/or to that of a control gene, such as, without limitation, β-Actin and PBDG (porphobilinogen deaminase) to determine the relative abundance of a given cDNA product of a given RNA in a RNA population. Products and reagents for the fluorescent hydrolysis probe assay are readily available commercially, for instance from Applied Biosystems.

Other detection reagents are commonly referred to as “Scorpions” and are described in U.S. Pat. Nos. 6,326,145 and 5,525,494. These reagents include one or more molecules comprising a tailed primer and an integrated signaling system. The primer has a template binding region and a tail comprising a linker and a target binding region. The target binding region in the tail hybridizes to complementary sequence in an extension product of the primer. This target specific hybridization event is coupled to a signaling system wherein hybridization leads to a detectable change. In PCR reactions the target binding region and the tail region are advantageously arranged such that the tail region remains single stranded, i.e. uncopied. Thus the tail region is non-amplifiable in the PCR amplification products. The linker comprises a blocking moiety which prevents polymerase mediated chain extension on the primer template.

Equipment and software also are readily available for controlling and monitoring amplicon accumulation in PCR and QRT-PCR including the Smart Cycler thermocylcer commercially available from Cepheid of Sunnyvale, Calif., and the ABI Prism 7700 Sequence Detection System, commercially available from Applied Biosystems.

In the preferred RT-PCR reactions, the amounts of certain reverse transcriptase and the PCR reaction components are atypical in order to take advantage of the faster ramp times of some thermal cyclers. Specifically, the primer concentrations are very high.

Typical gene-specific primer concentrations for reverse transcriptase reactions are less than about 20 nM. To achieve a rapid reverse transcriptase reaction on the order of one to two minutes, the reverse transcriptase primer concentration was raised to greater than 20 nM, preferably at least about 50 nM, and typically about 100 nM. Standard PCR primer concentrations range from 100 nM to 300 nM. Higher concentrations may be used in standard PCR reactions to compensate for Tm variations. However, for purposes herein, the referenced primer concentrations are for circumstances where no Tm compensation is needed. Proportionately higher concentrations of primers may be empirically determined and used if Tm compensation is necessary or desired. To achieve rapid PCR reactions, the PCR primer concentrations typically are greater than 250 nM, preferably greater than about 300 nM and typically about 500 nM.

Preferred primer/probe sets for both mammaglobin and CK19 are provided. The requirements for such a primer/probe combination is that it is able to identify a clinically significant quantity of CK19 mRNA, while not detecting a large quantity of genomic DNA. These primers and probes are useful for any applications for the specific detection of CK19 mRNA. Unexpectedly, this subset of primer/probe combinations proved significantly superior to the other combinations tested. Cytokeratin 19 has 4 pseudogenes that align with about 86-91% identity. These pseudogenes reside on chromosomes 4, 6, and 12. Assay design was restricted by having to incorporate an exon-intron junction so that CK19 DNA is not efficiently amplified and detected.

In the case of mammaglobin, the following are found to provide optimal results:

MG forward primer

(SEQ ID NO: 9)

AGTTGCTGATGGTCCTCATGC
MG reverse primer

(SEQ ID NO: 10)

ATCACATTCTCCAATAAGGGGCA
MG probe

(SEQ ID NO:11)

Fam-CCCTCTCCCAGCACTGCTACGCA-BHQ1-TT

In the case of CK19, the following are found to provide optimal results:

CK19 forward primer

(SEQ ID NO: 12)

CACCCTTCAGGGTCTTGAGATT
CK19 reverse primer

(SEQ ID NO: 13)

TCCGTTTCTGCCAGTGTGTC
CK19 probe

(SEQ ID NO: 14)

Q570-ACAGCTGAGCATGAAAGCTGCCTT-BHQ2-TT

Where these primer/probe sets are used, the following primer/probe set is optimal as a control to amplify and detect PBGD.

PBGD forward primer

(SEQ ID NO: 15)

GCCTACTTTCCAAGCGGAGCCA
PBGD reverse primer

(SEQ ID NO: 16)

TTGCGGGTACCCACGCGAA
PBGD probe

(SEQ ID NO: 17)

Q670-AACGGCAATGCGGCTGCAACGGCGGAA-BHQ2

Additional primers, probes and combinations thereof are provided in the Examples herein.

Commercially used diagnostics also preferably employ one or more internal positive controls that confirm the operation of a particular amplification reaction for a negative result. Potential causes of false negative results that must be controlled for in an RT-PCR reaction include: inadequate RNA quantity, degradation of RNA, inhibition of RT and/or PCR and experimenter error. In the case of gene expression assays, it is preferable to utilize a gene that is constitutively expressed in the tissue of interest. PBGD (SEQ ID NO: 7) is a gene that is commonly used as an internal control due to several factors: it contains no know pseudogenes in humans, it is constitutively expressed in human tissues and it is expressed at a relatively low level and therefore is less likely to cause inhibition of the amplification of target sequences of interest. Use of PBGD as a control minimizes or eliminates reporting erroneous results arising from all potential sources of false negative results.

In the commercialization of the described methods for QRT-PCR certain kits for detection of specific nucleic acids are particularly useful. In one embodiment, the kit includes reagents for amplifying and detecting Markers. Optionally, the kit includes sample preparation reagents and or articles (e.g., tubes) to extract nucleic acids from lymph node tissue. The kits may also include articles to minimize the risk of sample contamination (e.g., disposable scalpel and surface for lymph node dissection and preparation).

In a preferred kit, reagents necessary for the one-tube QRT-PCR process described above are included such as reverse transcriptase, a reverse transcriptase primer, a corresponding PCR primer set (preferably for Markers and controls), a thermostable DNA polymerase, such as Taq polymerase, and a suitable detection reagent(s), such as, without limitation, a scorpion probe, a probe for a fluorescent hydrolysis probe assay, a molecular beacon probe, a single dye primer or a fluorescent dye specific to double-stranded DNA, such as ethidium bromide. The primers are preferably in quantities that yield the high concentrations described above. Thermostable DNA polymerases are commonly and commercially available from a variety of manufacturers. Additional materials in the kit may include: suitable reaction tubes or vials, a barrier composition, typically a wax bead, optionally including magnesium; reaction mixtures (typically 10×) for the reverse transcriptase and the PCR stages, including necessary buffers and reagents such as dNTPs; nuclease- or RNase-free water; RNase inhibitor; control nucleic acid (s) and/or any additional buffers, compounds, co-factors, ionic constituents, proteins and enzymes, polymers, and the like that may be used in reverse transcriptase and/or PCR stages of QRT-PCR reactions. Optionally, the kits include nucleic acid extraction reagents and materials.

The following non-limiting examples help to further describe the invention. All documents cited herein are hereby incorporated herein by reference.

### EXAMPLES

### Real-Time PCR

Examples in the present invention are based on the use of real-time PCR. In real-time PCR the products of the polymerase chain reaction are monitored in real-time during the exponential phase of PCR rather than by an end-point measurement. Hence, the quantification of DNA and RNA is much more precise and reproducible. Fluorescence values are recorded during every cycle and represent the amount of product amplified to that point in the amplification reaction. The more templates present at the beginning of the reaction, the fewer number of cycles it takes to reach a point in which the fluorescent signal is first recorded as statistically significant above background, which is the definition of the (Ct) values. The concept of the threshold cycle (Ct) allows for accurate and reproducible quantification using fluorescence based RT-PCR. Homogeneous detection of PCR products are preferably performed based on: (a) double-stranded DNA binding dyes (e.g., SYBR Green), (b) fluorogenic probes (e.g., TaqMan® probes, Molecular Beacons), and (c) direct labeled primers (e.g., Amplifluor primers). Suitable methods are also described in U.S. patent application Ser. No. 10/427,243.

### Example 1

### Two Gene Identification of Breast Cancer Cells in SLNs

The presence of axillary lymph node (ALN) metastasis is the most important prognostic factor for breast cancer patients. SLN status is highly predictive of overall ALN involvement. SLN-positive patients have historically undergone ALN dissection in a second surgery. Intraoperative SLN analysis methods have been implemented to reduce the cost and complications of second surgeries, but these methods suffer from poor and variable sensitivity and a lack of standardization. The following example shows the feasibility of RT-PCR as the basis for improving the intraoperative diagnosis of clinically relevant SLN metastasis.

Methods: Eight molecular markers, including mammaglobin, were identified from a genome-wide gene expression analysis of breast and other tissues. Alternate serial sections of SLN from 254 breast cancer patients were analyzed by permanent section H&E or quick frozen for RNA extraction. Blinded SLN cDNAs were analyzed by quantitative RT-PCR. PCR signal was compared to H&E results on a patient basis. Using multivariate receiver operating characteristic (ROC) analysis, PCR cut-offs were selected that optimally correlated with H&E results.

Patient Samples: SLN RNA samples were obtained from a clinical endpoint PCR study of mammaglobin in lymph nodes of breast cancer patients at East Carolina University. Lymph node RNA was derived from half of the original node. Sample quality was assessed by Agilent, spectroscopy and housekeeping gene PCR analysis. Patients for which there were samples that were deemed of poor quality and were considered PCR negative for breast were removed from the study. All SLNs from 254 patients were included in this study.

Marker Validation: Seven Markers, including mammaglobin, were identified from the literature and internal bioinformatics methods and subsequently validated on primary breast tissue samples. PBGD and β-actin were employed as housekeeping genes. Lymph node cDNA was analyzed by quantitative PCR utilizing TaqMan® chemistry on an ABI Prism 7900HT sequence detection system. Data were reported in Ct. A Ct is defined as the cycle at which a statistically significant increase in normalized reporter emission is seen. Mammaglobin primers (SEQ ID NO: 18 and SEQ ID NO: 19) were synthesized by Invitrogen Corp. (Carlsbad, Calif.) and the mammaglobin TaqMan® probe (SEQ ID NO:20) from Epoch Biosciences (San Diego, Calif.). CK19 primers (SEQ ID NO:21 and SEQ ID NO:22) and the TaqMan® probe (SEQ ID NO:23). B726 primers (SEQ ID NO:24 and SEQ ID NO:25) and the TaqMan® probe (SEQ ID NO:26). B305D primers (SEQ ID NO:27 and SEQ ID NO:28) were synthesized at Invitrogen Corp and the probe (SEQ ID NO:29) by Applied Biosystems, Inc. PIP primers (SEQ ID NO:30 and SEQ ID NO:31) and the TaqMan® probe (SEQ ID NO:32). PDEF primers (SEQ ID NO:33 and SEQ ID NO:34) and the TaqMan® probe (SEQ ID NO:35). GABA primers (SEQ ID NO:36 and SEQ ID NO:37) and the TaqMan® probe (SEQ ID NO:38). PBGD primers (SEQ ID NO:39 and SEQ ID NO:40) were synthesized by QIAGEN Operon (Alameda, Calif.), and the probe (SEQ ID NO:41) by Synthegen, LLC (Houston, Tex.). For all TaqMan® probes, carboxyfluorescein (FAM) and carboxytetramethylrhodamine TAMRA) were used as the dye and quencher pair.

SEQ ID NO: 18

CAAACGGATG AAACTCTGAG CAATGTTGA

SEQ ID NO: 19

TCTGTGAGCC AAAGGTCTTG CAGA

SEQ ID NO: 20

6-FAM-tgtttatgca attaatatat gacagcagtc tttgtg-
TAMRA

SEQ ID NO: 21

AGATGAGCAGGTCCGAGGTTA

SEQ ID NO: 22

CCTGATTCTGCCGCTCACTATCA

SEQ ID NO: 23

FAM-ACCCTTCAGGGTCTTGAGATTGAGCTGCA-TAMRA

SEQ ID NO: 24

GCAAGTGCCAATGATCAGAGG

SEQ ID NO: 25

ATATAGACTCAGGTATACACACT

SEQ ID NO: 26

FAM TCCCATCAGAATCCAAACAAGAGGAAG

SEQ ID NO: 27

TCTGATAAAG GCCGTACAAT G

SEQ ID NO: 28

TCACGACTTG CTGTTTTTGC TC

SEQ ID NO: 29

6-FAM-ATCAAAAAACA AGCATGGCCTCA CACC-TAMRA

SEQ ID NO: 30

GCTTGGTGGTTAAAACTTACC

SEQ ID NO: 31

TGAACAGTTCTGTTGGTGTA

SEQ ID NO: 32

FAM-CTGCCTGCCTATGTGACGACAATCCGG-TAMRA

SEQ ID NO: 33

GCCGCTTCATTAGGTGGCTCAA

SEQ ID NO: 34

AGCGGCTCAGCTTGTCGTAGTT

SEQ ID NO: 35

AAGGAGAAGGGCATCTTCAAAATTGAGGACTCAGC

SEQ ID NO: 36

CAATTTTGGTGGAGAACCCG

SEQ ID NO: 37

GCTGTCGGAGGTATATGGTG

SEQ ID NO: 38

FAM CATTTCAGAGAGTAACATGGACTACACA TAMRA

SEQ ID NO: 39

CTGCTTCGCTGCATCGCTGAAA

SEQ ID NO: 40

CAGACTCCTCCAGTCAGGTACA

SEQ ID NO: 41

FAM-CCTGAGGCACCTGGAAGGAGGCTGCAGTGT-TAMRA

Data Analysis: Samples were unblinded at the conclusion of the PCR testing. H&E, IHC, recurrence and additional pathological data were made available at such time. Ct cut-offs were established for determination of positive lymph node status using multivariate receiver operating characteristic (ROC) analysis and visual observations. Discrepant resolution (based on pathology reports) was carried out for all false-negative and false-positive results. Presumptive positive samples (samples that likely represent true positives missed by standard pathology due to inadequate nodal sampling) were identified based on the following criteria: at least four molecular markers positive, with at least one of the markers strongly positive (Ct at least 5 cycles below the single marker assay cut-offs).

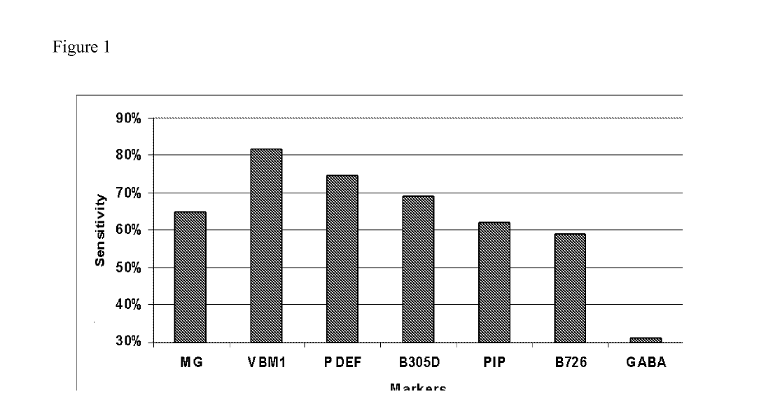

The results are presented in FIG. 1 and Tables 1-2. In FIG. 1, VBM1 is CK19.

TABLE 1
Optimal Performance with Varying Numbers of Markers

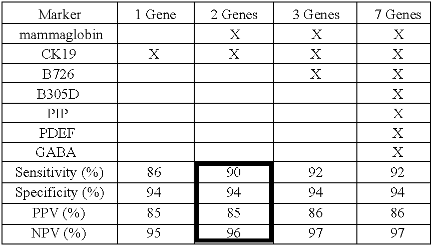

TABLE 2
Correlation of Two-Gene Signature
with Histology FFPE H&E w/o IHC

| | | +ve | −ve |
|---|---|---|---|
| Assay | +ve | 64 | 11 |
| markers | −ve | 7 | 172 |
| | | 71 | 183 |

### In Table 2 Sensitivity is 90%, Specificity is 93%, PPV is 84% and NPV is 96%.

TABLE 3
Identification of Presumptive Positive Samples

| | | | Markers | Markers | Presumptive |
|---|---|---|---|---|---|
| Sample | MG | CK19 | Positive | Strongly Positive | Positive |
| 1 | | + | 1 | 0 | |
| 2 | ++ | ++ | 7 | 6 | + |
| 3 | + | + | 5 | 0 | |
| 4 | ++ | + | 7 | 3 | + |
| 5 | + | + | 6 | 0 | |
| 6 | | ++ | 5 | 4 | + |
| 7 | ++ | ++ | 7 | 3 | + |
| 8 | ++ | | 2 | 1 | |
| 9 | ++ | | 2 | 1 | |
| 10 | ++ | ++ | 4 | 3 | + |
| 11 | ++ | | 4 | 1 | + |

+ PCR Positive (Ct ≦ cut-off)
++ Strongly PCR Positive (>5 cycles below Ct cut-off)
Presumptive Positive PCR Positive with ≧4 Markers and strongly PCR Positive with at least 1 Marker

TABLE 4
Correlation of Two-Gene Signature with Presumptive
Positivity FFPE H&E (+) or Presumptive (+)

| | | +ve | −ve |
|---|---|---|---|
| Assay | +ve | 71 | 5 |
| markers | −ve | 7 | 171 |
| | | 78 | 176 |

In Table 4 Sensitivity is 91%, Specificity is 97%, PPV is 93% and NPV is 96%.

Results: A two-gene assay (mammaglobin and CK19) detected clinically actionable metastasis (H&E-positive in the absence of IHC) with 90% sensitivity and 93% specificity. Addition of a third gene had minimal impact on overall performance.

As part of ongoing efforts to characterize genes to achieve better sensitivity and specificity, PDEF and CK19 assays were run on these lymph node samples. The CK19+MG combination of markers increased the sensitivity of the assay further as shown below in Table 5.

TABLE 5
MG + B305D Marker combination

Permanent Section
H&E > 0.2 mm

| | | Positive | Negative |
|---|---|---|---|
| MG + | Positive | 53 | 11 |
| B305D | Negative | 18 | 172 |
| | | 71 | 183 |

Sensitivity = 75%
Specificity = 94%
PPV = 83%
NPV = 91%

TABLE 6
CK19 + MG marker combination

Permanent Section
H&E > 0.2 mm

| | | Positive | Negative |
|---|---|---|---|
| CK19 + | Positive | 64 | 11 |
| MG | Negative | 7 | 172 |
| | | 71 | 183 |

Sensitivity = 90%
Specificity = 94%
PPV = 85%
NPV = 96%

These data not only showed that CK19 increased the sensitivity of the assay; it also was the primary marker with Mammaglobin being the complementing marker. This marker combination improves assay performance

Conclusions: This study demonstrates the utility of RT-PCR as the basis of an intraoperative assay for detecting clinically actionable breast metastasis with Mammaglobin/CK19 expression closely correlating with standard H&E detection of SLN metastasis, demonstrating that two-gene (one breast cancer specific and one non-tissue specific) molecular signature analysis detects clinically relevant metastasis in breast SLNs. Thus, the test has the potential to significantly reduce second surgeries for patients undergoing SLN biopsies.

### Example 2

### Optimal Primers and Probes for the Specific Detection of Mammaglobin, CK19 and PBGD mRNA

### Mammaglobin Primers and Probes

The ability of Tth polymerase to provide adequate strand displacement and nuclease activities for a rapid assay was determined and primer and probes optimized for the appropriate assay conditions. The first set of primers/probes were specific for Exons 2 and 3. Experiments were conducted with and without Sybr Green to distinguish between successful amplification and detection. Optimization of divalent cation concentrations and addition of Magnesium (MgCl₂) to Manganese (MnSO₄) was performed to improve RT and amplification. One of these experiments also showed no significant difference between MnCl₂ and MnSO₄. These experiments lead to optimal assays utilizing two divalent cations—manganese (MnSO₄) for RT and magnesium (MgCl₂) for PCR at 2.5 mM and 3.5 mM respectively.

The first design generated a 105 bp amplicon and was redesigned in the same region to yield a smaller amplicon of 96 bp. The first and second designs worked very well but showed really high signals and spillover into Cy3 channel from the Fam channel.

An assay was redesigned for Mammaglobin spanning exons 1 and 2 because designs across exons 2 and 3 were in AT rich regions and might present problems during multiplexing efforts by limiting flexibility in cycling temperatures. Two probes were designed in the same region. Both probes were tested in the Fam, Cy3, and Cy5 channels. The new design was validated with and without Sybr Green to ensure that there was no inhibition during RT due to the presence of the probe.

### Mammaglobin Design History

SEQ ID NO: 18

| | |
|---|---|
| CAAACGGATGAAACTCTGAGCAATGTTGA | Exons 2-3 |

SEQ ID NO: 19

| | |
|---|---|
| TCTGTGAGCCAAAGGTCTTGCAGA | 105 bp |

SEQ ID NO: 20

| | |
|---|---|
| TGTTTATGCAATTAATATATGACAGCAGTCTTTGT | Product |

SEQ ID NO: 42

| | |
|---|---|
| CGGATGAAACTCTGAGCAATGTTGA | Exons 2-3 |

SEQ ID NO: 43

| | |
|---|---|
| GAGCCAAAGGTCTTGCAGAAAGT | 96 bp |

SEQ ID NO: 44

| | |
|---|---|
| TGTTTATGCAATTAATATATGACAGCAGTCTTTGTG | Product |

SEQ ID NO: 9

| | |
|---|---|
| AGTTGCTGATGGTCCTCATGC | Exons 1-2 |

SEQ ID NO: 10

| | |
|---|---|
| ATCACATTCTCCAATAAGGGGC | 82 bp |

SEQ ID NO: 45

| | |
|---|---|
| GCACTGCTACGCAGGCTCTGGC | Product |

SEQ ID NO: 11

| | |
|---|---|
| CCCTCTCCCAGCACTGCTACGCA | Product |

Based on data collected using both probe designs, SEQ ID NO: 48 was picked as the final design for the exons1-2 region.
Mammaglobin Final Design from Singlex Testing

Following experimentation validating the new designs, Mammaglobin was put in the Fam channel using the following sequences as primers and probe.

| | |
|---|---|
| SEQ ID NO: 9 | AGTTGCTGATGGTCCTCATGC |
| SEQ ID NO: 10 | ATCACATTCTCCAATAAGGGGCA |
| SEQ ID NO: 11 | Fam-CCCTCTCCCAGCACTGCTACGCA-BHQ1 |

Once it was determined that the hydrolysis probe assay was suitable for Tth polymerase, the assay was tested for B305D and CK19 as well.

### CK19

First Oligonucleotide Set

The initial design tested included a junction-specific PCR primer into the design, as this appeared to best discriminate between CK19 and its pseudogenes. The primer and dual-labeled hydrolysis probe sequences tested for this design are shown below:

SEQ ID NO: 21

| | |
|---|---|
| Forward primer | AGATGAGCAGGTCCGAGGTTA |

SEQ ID NO: 46

| | |
|---|---|
| Reverse primer | GCAGCTTTCATGCTCAGCTGT |

SEQ ID NO: 23

| | |
|---|---|
| Probe (5′FAM/3′BHQ) | ACCCTTCAGGGTCTTGAGATTGAGCTGCA |

As shown below in Table 7, this design was demonstrated to strongly cross-react with genomic DNA:

TABLE 7

| | | Adjusted | Probe | SYBR | Adjusted SYBR |
|---|---|---|---|---|---|
| Target | Probe Ct | Ct | Fluorescence | Green Ct | Green Ct |
| 15,500 copies DNA | 26.6 | 23.9 | 225.1 | 22.8 | 20.1 |
| 100,000 copies RNA | 25.3 | 25.3 | 252.0 | 23.0 | 23.0 |

When adjusted to account for differences in target concentration, the probe Ct's observed with DNA and RNA were essentially identical. In addition, the end-point fluorescence from the hydrolysis probes was also essentially identical. Taken together, these results demonstrate poor primer AND probe specificity for RNA versus DNA. SYBR Green signal from separate reactions suggests that amplification is actually superior for DNA target versus RNA target, possibly due to amplification of multiple pseudogene sequences or inefficiencies in the conversion of RNA to DNA during one-step RT-PCR.

Second Oligonucleotide Set

The next design tested included a junction-specific probe and primers in separate exons. The primer and dual-labeled hydrolysis probe sequences tested for this design are shown below:

(SEQ ID NO: 47)

| | |
|---|---|
| Forward | CACCCTTCAGGGTCTTGAGA |
| primer | |

(SEQ ID NO: 48)

| | |
|---|---|
| Reverse | TCCGTTTCTGCCAGTGTGTC |
| primer | |

(SEQ ID NO: 49)

| | |
|---|---|
| Probe | (5′FAM/3′BHQ) GCTGAGCATGAAAGCTGCCTTGGA |

In this case, the adjusted Ct for DNA was 2.5 cycles higher than for RNA, demonstrating some level of specificity for RNA versus DNA. The fact that the Ct difference between RNA and DNA is greater for the probe than for SYBR Green (2.5 cycles versus 0.2 cycles) suggests that the specificity improvement is due to both the primers and the probe. Compared to the previous design, the improvement in primer specificity (SYBR Green Ct) is 3.1 cycles (0.2 cycles versus −2.9 cycles). The improvement in probe specificity is supported by the two-fold increase in fluorescence for RNA relative to DNA (Table 8). While this increase in specificity is desirable, it may not be adequate to confidently discriminate RNA signal from DNA signal.

TABLE 8

| | | | | SYBR | Adjusted |
|---|---|---|---|---|---|
| | Probe | Adjusted | Probe | Green | SYBR |
| Target | Ct | Ct | Fluorescence | Ct | Green Ct |
| 15,500 copies | 29.5 | 26.8 | 223.9 | 23.2 | 20.5 |
| DNA | | | | | |
| 100,000 copies | 24.3 | 24.3 | 453.7 | 20.3 | 20.3 |
| RNA | | | | | |
| Third oligo- | | | | | |
| nucleotide set | | | | | |

Additional primers and probes were designed to further improve specificity for RNA. Additional designs were made in the same region with minor modifications in the location and length of the primers and probes. The primer and dual-labeled hydrolysis probe sequences tested are shown below in Table 9.

| | |
|---|---|
| Forward primers | |
| (SEQ ID NO: 47) | CACCCTTCAGGGTCTTGAGA |
| (SEQ ID NO: 50) | CACCCTTCAGGGTCTTGAGAT |
| (SEQ ID NO: 12) | CACCCTTCAGGGTCTTGAGATT |
| (SEQ ID NO: 51) | ACCCTTCAGGGTCTTGAGATTG |
| (SEQ ID NO: 52) | ACCCTTCAGGGTCTTGAGATTGA |
| Reverse primers | |
| (SEQ ID NO: 13) | TCCGTTTCTGCCAGTGTGTC |
| (SEQ ID NO: 53) | CTCCGTTTCTGCCAGTGTGT |
| Probes (5′FAM/3′BHQ) | |
| (SEQ ID NO: 49) | GCTGAGCATGAAAGCTGCCTTGGA |
| (SEQ ID NO: 14) | ACAGCTGAGCATGAAAGCTGCCTT |

TABLE 9

| | Forward | Reverse | | Adjusted | RNA/DNA Probe |
|---|---|---|---|---|---|
| | primer | Primer | Probe | RNA/DNA Probe | Fluorescence |
| Cond. | SEQ ID NO: | SEQ ID NO: | SEQ ID NO: | Ct Difference | Ratio |
| A | 47 | 13 | 49 | 2.5 | 2.0 |
| B | 12 | 13 | 49 | 3.7 | 3.6 |
| C | 50 | 13 | 49 | −0.6 | 1.3 |
| D | 51 | 13 | 49 | 2.5 | 3.4 |
| E | 52 | 13 | 49 | 2.5 | 2.6 |
| F | 47 | 53 | 49 | 1.4 | 2.0 |
| G | 12 | 53 | 49 | 4.1 | 3.7 |
| H | 50 | 53 | 49 | −0.8 | 1.3 |
| I | 51 | 53 | 49 | 4.6 | 3.8 |
| J | 52 | 53 | 49 | 0.6 | 2.8 |
| K | 47 | 53 | 14 | 2.2 | 4.1 |
| L | 12 | 53 | 14 | 3.6 | 10.3 |
| M | 50 | 53 | 14 | −0.8 | 2.1 |
| N | 51 | 53 | 14 | 4.4 | 10.9 |
| O | 52 | 53 | 14 | 4.1 | 8.4 |
| P | 12 | 13 | 14 | Not tested | |
| Q | 51 | 13 | 14 | Not tested | |
| R | 52 | 13 | 14 | Not tested | |

Compared to the condition described above, (Condition A), several conditions demonstrated an improvement in either adjusted RNA/DNA probe Ct difference or RNA/DNA probe fluorescence ratio. The optimal conditions were L, N and O. All of these conditions demonstrated Ct differences of at least 3.6 cycles and fluorescence rations of at least 8. All three conditions demonstrate enough signal discrimination to all elimination of DNA detection through minimal manipulation of the fluorescent cut-offs used to define positivity. Conditions B, D, G, I and K all have Ct differentials >2.2 cycles and fluorescence ratios of >3.4, suggesting a reasonable potential to utilize these combinations to resolve between RNA and DNA. Though not tested, conditions P, Q and R (similar, respectively, to L, N, and O, except utilizing reverse primer SEQ ID NO: 13) would also be predicted to lead to optimal performance.

Conditions L and P were tested to demonstrate the ability to further increase the specificity for RNA by modifying the assay fluorescence cut-offs. The primer and dual-labeled hydrolysis probe sequences tested for this example are shown below:

### Forward Primer

### (SEQ ID NO:12) CACCCTTCAGGGTCTTGAGATT

| | |
|---|---|
| Reverse primers | |
| (SEQ ID NO: 13) | TCCGTTTCTGCCAGTGTGTC |
| (SEQ ID NO: 53) | CTCCGTTTCTGCCAGTGTGT |
| Probe (5′Q570/3′BHQ2) | |
| (SEQ ID NO: 14) | ACAGCTGAGCATGAAAGCTGCCTT |

As shown in Table 10, by increasing the cut-offs from a current set-point of approximately 1.5% of maximum fluorescence to a level of 6-7% of maximum fluorescence, no Ct was observed for DNA out to 40 cycles, while the Ct for RNA increased by only about 2 cycles. This type of minor modification to cut-offs is predicted to lead to optimal performance for condition L, N, O, P, Q, and R, at a minimum.

TABLE 10

| | Forward primer | Reverse Primer | Probe | | |
|---|---|---|---|---|---|
| | SEQ ID | SEQ ID | SEQ ID | | |
| Condition | NO: | NO: | NO: | RNA Ct | DNA Ct |
| L | 12 | 53 | 14 | 25.9 | 40.0 |
| P | 12 | 13 | 14 | 25.6 | 40.0 |

PBGD primers/probes

| | | |
|---|---|---|
| SEQ ID NO: 15 | GCCTACTTTCCAAGCGGAGCCA | Exons 1-2 |
| SEQ ID NO: 54 | ACCCACGCGAATCACTCTCA | 83 bp |
| SEQ ID NO: 17 | AACGGCAATGCGGCTGCAACGGCGGAA | Product |
| SEQ ID NO: 55 | CAAGCGGAGCCATGTCTGG | Exons 1-2 |
| SEQ ID NO: 54 | ACCCACGCGAATCACTCTCA | 93 bp |
| SEQ ID NO: 17 | AACGGCAATGCGGCTGCAACGGCGGAA | Product |

Both designs performed equally well but the longer product was chosen as the final design. PBGD was put in the Cy5 channel. This was done to make sure that any positive result of the internal control would not be caused due to spillover from the other channel. PBGD Final Design from Singlex testing.

| | |
|---|---|
| SEQ ID NO: 55 | CAAGCGGAGCCATGTCTGG |
| SEQ ID NO: 54 | ACCCACGCGAATCACTCTCA |
| SEQ ID NO: 17 | Q670-AACGGCAATGCGGCTGCAACGGCGGAA-BHQ2 |

### Multiplex Testing

Following singlex testing, the above chosen designs were tested in multiplex with Mammaglobin in Fam, CK19 in Cy3 and PBGD in Cy5 channels in the presence and absence of Sybr Green. It was observed that the No Template reactions were generating much lower Cts in the multiplex. Upon further experimentation with different primer-probe combinations, it was seen that there was a 3′ dimerization between the PBGD lower and CK19 upper primers. With the absence of one of these two primers in the multiplex mix, the no template reactions came up at much higher Cts.

In order to minimize primer interactions, the following primers were chosen for the final multiplexed assay.

| | |
|---|---|
| MG forward primer | |
| (SEQ ID NO: 9) | AGTTGCTGATGGTCCTCATGC |
| MG reverse primer | |
| (SEQ ID NO: 10) | ATCACATTCTCCAATAAGGGGCA |
| MG probe | |
| (SEQ ID NO: 11) | Fam-CCCTCTCCCAGCACTGCTACGCA- |
| | BHQ1-TT |
| CK19 forward primer | |
| (SEQ ID NO: 12) | CACCCTTCAGGGTCTTGAGATT |
| CK19 reverse primer | |
| (SEQ ID NO: 13) | TCCGTTTCTGCCAGTGTGTC |
| CK19 probe | |
| (SEQ ID NO: 14) | Q570-ACAGCTGAGCATGAAAGCTGCCTT- |
| | BHQ2-TT |
| PBGD forward primer | |
| (SEQ ID NO: 15) | GCCTACTTTCCAAGCGGAGCCA |
| PBGD reverse primer | |
| (SEQ ID NO: 16) | TTGCGGGTACCCACGCGAA |
| PBGD probe | |
| (SEQ ID NO: 17) | Q670-AACGGCAATGCGGCTGCAACGGCGGAA- |
| | BHQ2 |

During final primer selection for CK19, comparison of primers, probes, and cycling conditions was done. These experiments showed that pseudogenes would not be detected in the Cy3 channel due to 10-fold discrimination in fluorescence levels between CK19 and its pseudogenes. Following feasibility studies, a separate experiment was designed to look at amplification of pseudogenes using genomic DNA. In all cases, it was evident that the pseudogenes were being amplified and not genomic DNA because the amplified product was of the correct length in all cases and did not show amplification of the intron. This experiment was run with and without Sybr Green to differentiate between amplification and detection. Different concentrations of genomic DNA were used as template along with a no template control. Reactions were run with only PCR as well as RT-PCR. Hydrolysis probe chemistry in the Cy3 channel did not pick up the pseudogenes.

However, it is evident from the SYBR data that the pseudogenes are being amplified but are not being detected using the hydrolysis probe chemistry in the Cy3 channel even at a concentration of 10⁶ copies. All products were run on gels to confirm results. In all cases where a template was used, a band of the correct size (81 bp) was seen including reactions without SYBR where no Ct was detected. This confirmation is essential in order to make sure that the absence of signal in the Cy3 channel is not because of the absence of amplification but because of discrimination due to the hydrolysis probe chemistry.

TABLE 11
Comparison of CK19 reactions with and without SYBR Green

| | | |
|---|---|---|
| Genomic | SYBR | Q570 |
| DNA | Ct | Ct |

PCR

| | | |
|---|---|---|
| 1.00E+06 | 22.99 | 40.00 |
| 1.00E+05 | 23.53 | 40.00 |
| 1.00E+04 | 26.65 | 40.00 |
| 1.00E+03 | 30.02 | 40.00 |
| NT | 37.85 | 40.00 |

RAPID TM RT-PCR

| | | |
|---|---|---|
| 1.00E+06 | 22.61 | 40.00 |
| 1.00E+05 | 23.07 | 40.00 |
| 1.00E+04 | 25.85 | 40.00 |
| 1.00E+03 | 29.58 | 40.00 |
| NT | 37.42 | 40.00 |

### Example 3

### Rapid Assay Testing of Samples Purified by Standard Methods Samples

RNA was isolated from breast lymph nodes by a standard Trizol method. RNA was quantitated by absorbance at 260 nm. All RNAs were diluted to 200 ng/μl. RNA quality was determined by two-step RT-PCR using the housekeeping genes PBGD and β-actin. Samples were considered of adequate quality if both housekeeping genes gave signals within 3 cycles of the mean signal across all samples tested. The final set of samples tested represented 487 lymph node samples from 251 patients.

### One-Step RT-PCR Testing

The RNA samples described above were amplified utilizing rapid, real-time, one-step RT-PCR containing primers and probes for mammaglobin (MG), keratin 19 (CK19) and PBGD. The primer and probe sequences utilized were as follows:

| | |
|---|---|
| MG forward primer | |
| (SEQ ID NO: 9) | AGTTGCTGATGGTCCTCATGC |
| MG reverse primer | |
| (SEQ ID NO: 10) | ATCACATTCTCCAATAAGGGGCA |
| MG probe | |
| (SEQ ID NO: 11) | Fam-CCCTCTCCCAGCACTGCTACGCA- |
| | BHQ1-TT |
| CK19 forward primer | |
| (SEQ ID NO: 12) | CACCCTTCAGGGTCTTGAGATT |
| CK19 reverse primer | |
| (SEQ ID NO: 13) | TCCGTTTCTGCCAGTGTGTC |
| CK19 probe | |
| (SEQ ID NO: 14) | Q570-ACAGCTGAGCATGAAAGCTGCCTT- |
| | BHQ2-TT |
| PBGD forward primer | |
| (SEQ ID NO: 15) | GCCTACTTTCCAAGCGGAGCCA |
| PBGD reverse primer | |
| (SEQ ID NO: 16) | TTGCGGGTACCCACGCGAA |
| PBGD probe | |
| (SEQ ID NO: 17) | Q670-AACGGCAATGCGGCTGCAACGGCGGAA- |
| | BHQ2 |

### RT-PCR Amplification Conditions

One μl (200 ng) of RNA from each sample was amplified in a 25 μl reaction containing the following components: 50 mM Bicine/KOH, pH 8.2, 3.5 mM MgCl₂, 2.5 mM manganese sulphate, 115 mM Potassium Acetate, 12 mM potassium chloride, 6 mM sodium chloride, 0.8 mM sodium phosphate, 10% v/v Glycerol, 0.2 mg/ml BSA, 150 mM Trehalose, 0.2% v/v Tween 20, 0.016% v/v Triton X-100 50 mM Tris-Cl pH 8, 0.2 mM dATP, 0.2 mM dCTP, 0.2 mM dGTP, 0.2 mM TTP, 0.08% v/v ProClin 300, 5 units Tth polymerase (a recombinant DNA polymerase/reverse transcriptase cloned from Thermos thermophilis), 400 ng Antibody TP6-25.3, 450 nM each primer for MG and CK19, 300 nM each primer for PBGD and 200 nM each probe. The RT-PCR conditions were carried out on the Cepheid Smart Cycler II utilizing the following profile:

3 sec incubation at 95° C.
150 sec incubation at 63° C.
4 sec incubations at the following temperatures: 63.2° C., 63.4° C., 64.0° C., 64.5° C., 65.0° C., 65.5° C., 66.0° C., 66.5° C., 67.0° C., 67.5° C., 68.0° C., 68.5° C., 69.0° C., 69.5° C.
90 sec incubation at 70°
then 40 cycles of:
1 sec incubation at 95°
6 sec incubation at 60.0°

Fluorescent signal was monitored during the 60.00 in channels 1 (Fam), 2 (Q570) and 4 (Q670) of the Smart Cycler II utilizing the following threshold fluorescent values for a positive Ct: 30 for channel 1, 20 for channel 2, and 20 for channel 3. Ct values were determined for each sample for all three channels and then optimal cut-offs were determined to correlate assay signal with H&E-positivity in the absence of IHC. Five samples were determined to have unacceptable RNA quality as determined by the PBGD signal and were considered no test results. The following Table 12 summarizes the results of the 246 patients for which valid results were obtained:

H&E (+) without IHC

| | | (+) | (−) |
|---|---|---|---|
| Assay | + | 60 | 8 |
| markers | − | 7 | 171 |
| | | 67 | 179 |

Sensitivity = 90%
Specificity = 96%
PPV = 88%
NPV = 96%

### Example 4

### Rapid Assay Testing of Samples Purified with the Rapid Sample Prep Method Samples

RNA was isolated from breast lymph nodes utilizing an Omni homogenizer and disposable probe for homogenization, followed by purification of RNA with the RNeasy (Qiagen) kit reagents utilizing the following protocol:

### Homogenization

Determined the sample weight in milligrams, if not previously recorded. Placed a fresh piece of wax paper on the balance, tared, and weighed the sample.

Note: Nodes less than 30 mg do not provide sufficient tissue to test using the BLN assay.

Using a fresh scalpel, minced all tissue from the same node into pieces approximately 1 mm in diameter. Care taken to avoid contamination of the tissue during processing.

Added homogenization buffer to the homogenization tube (8 or 15 mL polypropylene culture tube, for homogenization buffer volume below 4 mL, use 8 mL tube, otherwise use 15 mL tube) used Table 13 to determine the required volume.

TABLE 13
Volume of Homogenization Buffer required

| Tissue | Homogenization |
|---|---|
| Weight (mg) | Buffer (mL) |
| 30-99 | 2 |
| 100-149 | 2 |
| 150-199 | 3 |
| 200-249 | 4 |
| 250-299 | 5 |
| 300-349 | 6 |
| 350-399 | 7 |
| 400-449 | 8 |
| 450-499 | 9 |
| 500-550 | 10 |
| >550 | See note below |

Note: Tissue of weight greater than 550 mg not adequately homogenized using the recommended system. The tissue was divided into equivalent parts prior to homogenization and each part should be homogenized, purified, and assayed as an individual specimen.
1. Using a clean forceps, transferred the tissue into the homogenization buffer.
2. Placed a new homogenization probe onto the manual homogenizer.
3. Homogenized each node completely.
4. Processed the homogenate as described in the RNA Purification section.
5. Disposed of the homogenization probe.
6. Stored any remaining homogenate at −65° C. or below.

### RNA Purification

Note: Multiple homogenates were processed in parallel using this procedure.
1. Mixed 400 μL of homogenate with 400 μL of 70% ethanol in a 4.5 mL tube by vortexing for 10 seconds.
2. For each sample, attached a VacValve onto a Vacuum Manifold, and a disposable VacConnector to each valve.
3. Attached a spin column on to the VacConnector, leaving the cap open.
4. Aliquotted the homogenate/ethanol mix from step 1 onto the column. The volume of homogenate/ethanol mix was based on the original tissue amount and is provided in 14.

TABLE 14

| Tissue | Volume of homogenate/ |
|---|---|
| Weight (mg) | ethanol mix (μL) |
| 30-39 | 700 |
| 40-49 | 500 |
| 50-59 | 400 |
| 60-69 | 350 |
| 70-79 | 300 |
| 80-89 | 250 |
| 90-99 | 225 |
| ≧100 | 200 |

5. Turned VacValves to the on position and apply vacuum (800-1000 mbars) until sample was filtered (approximately 30 seconds).
6. Stopped vacuum; add 700 μL of Wash Buffer 1 to the column. Started vacuum and allowed the solution to filter through the column. Stopped vacuum.
7. Added 700 μL of Wash Buffer 2 to the column. Started vacuum and allowed the solution to filter through the column. Stopped vacuum.
8. Removed each column from the Vacuum Manifold and placed into a 2 mL collection tube.
9. Centrifuged tube containing the spin columns for 30 sec at 13,200 RPM in a micro centrifuge.
10. Discarded the collection tube. Removed the column and put into a new fresh collection tube.
11. Added 50 μL of RNAase-free water directly to the filter membrane of column.
12. Centrifuged at 13,200 RPM for 30 sec in a microcentrifuge.
13. Discarded the column. Approximately 50 μL of eluted RNA solution was contained in the collection tube. Five μl of this solution was used per 25 μl reaction.

The final set of samples tested represented 30H&E-positive and 25H&E-negative axillary lymph nodes from sentinel lymph node-positive breast cancer patients.

One-step RT-PCR testing

Five μl of the eluted RNA was run in a 25 μl rapid, one-step RT-PCR reaction as described in Example 3, utilizing cut-offs derived from the patient samples tested in this Example, except that the cut-offs were normalized to account for the differences between the two sample preparation methods employed. Three samples were determined to have unacceptable RNA quality as determined by the PBGD signal and were considered no test results. The following Table 15 summarizes the results of the 52 nodes for which valid results were obtained:

H&E (+) without IHC

| | | (+) | (−) |
|---|---|---|---|
| Assay | + | 29 | 1 |
| markers | − | 0 | 22 |
| | | 29 | 23 |

Sensitivity = 100%
Specificity = 96%
PPV = 97%
NPV = 100%

Current reaction conditions include:

### Breast Lymph Node Assay Components

Master Mix

| | | Conc in | Conc | Final Conc |
|---|---|---|---|---|
| | | Master | Added | in 25 μl |
| Component | Unit | Mix | to MM | Rxn (from MM) |
| Bicine | mM | 125 | 125 | 50 |
| KOH | mM | 48 | 48 | 19.2 |
| Potassium Acetate | mM | 287.5 | 287.5 | 115 |
| D (+) Trehalose | mM | 375 | 375 | 150 |
| Tris-Cl pH 8 | mM | 135 | 125 | 50 |
| Albumin, Bovine | mg/ml | 0.5 | 0.5 | 0.2 mg/ml |
| MnSO4 | mM | 7.5 | 7.5 | 3.0 |
| MgCl2 | mM | 3.125 | 3.125 | 1.25 |
| Tween 20 | % | 0.5% | 0.5% | 0.2% |
| ProClin 300 | % | 0.08% | 0.08% | 0.08% |
| Glycerol | % | 15.0% | 15.0% | 6.0% |
| dNTP Mix | mM | 0.5 | 0.5 | 0.2 |
| CK19 Probe | nm | 500 | 500 | 200 |
| MgA Probe | nm | 500 | 500 | 200 |
| B305D Probe | nm | 500 | 500 | 200 |
| CK19 5′-3′ Primer | nm | 1125 | 1125 | 450 |
| CK19 3′-5′ Primer | nm | 1125 | 1125 | 450 |
| MgA 5′-3′ Primer | nm | 1125 | 1125 | 450 |
| MgA 3′-5′ Primer | nm | 1125 | 1125 | 450 |
| PBGS 5′-3′ Primer | nm | 750 | 750 | 300 |
| PBGS 3′-5′ Primer | nm | 750 | 750 | 300 |

| Tth Storage Buffer | | | | |
|---|---|---|---|---|
| | | Stock | Concentration in EM Bulk | |
| | Unit | Conc | | Additional |
| Tth Polymerase | Units | 5/μl | From Storage | (Suggested |
| TP6-25 Ab | mg | 1 mg | Buffer | EM formulation) |
| Glycerol | % | 50% | 6.5% | 3.5% |
| Tris-HCl | mM | 10 | 1.3 | 9.0 |
| KCl | mM | 300 | 39.0 | 0 |
| EDTA | mM | 0.1 | 0.01 | 0.0 |
| Triton X-100 | % | 0.1% | 0.01% | 0.0 |
| Dithiothreitol | mM | 1 | 0.1 | 0.0 |
| (DTT) | | | | |
| NaPO4 | mM | 20 | 2.6 | x |
| NaCl | mM | 150 | 19.5 | x |
| | | | 7.69230769 | |

We claim:

1. A method of diagnosing the presence or predicting the course of breast cancer comprising measuring the expression of a combination of Marker genes comprising at least one tissue-specific gene and at least one non-tissue-specific gene in a cell or tissue sample derived from a patient.

2. The method according to claim 1, wherein the tissue-specific gene is selected from the group consisting of mammaglobin (SEQ ID NO: 1), PIP (SEQ ID NO: 3), B305D (SEQ ID NO: 4), B726 (SEQ ID NO: 5), GABA (SEQ ID NO: 6) and PDEF (SEQ ID NO: 7).

3. The method according to claim 1, wherein the tissue-specific gene is mammaglobin (SEQ ID NO: 1).

4. The method according to claim 1, wherein the non-tissue-specific gene encodes a protein associated specifically with epithelial cells.

5. The method according to claim 4 wherein the gene is selected from the group consisting of CK19 (SEQ ID NO: 2), lumican, selenoprotein P, connective tissue growth factor, EPCAM, E-cadherin, and collagen, type IV, α-2.

6. The method according to claim 5 wherein the gene is CK19 (SEQ ID NO: 2).

7. The method according to claim 1 wherein the genes are mammaglobin (SEQ ID NO: 1) and CK19 (SEQ ID NO: 2).

8. The method according to claim 7 further comprising a control reaction measuring expression of a gene constitutively expressed in the sample.

9. The method according to claim 8 wherein the gene is PBGD (SEQ ID NO: 8).

10. The method according to claim 1 used for identifying patients at risk for metastasis.

11. The method of claim 1 used for detecting metastasis.

12. The method of claim 8 used for detecting breast cancer metastasis.

13. The method of claim 1 wherein all of the steps are conducted during the course of a surgical procedure.

14. The method according to claim 13, wherein expression is measured by conducting an intraoperative molecular diagnostic assay comprising the steps of: obtaining a lymph node tissue sample from a patient; analyzing the sample by nucleic acid amplification and detection; and determining if the presence of more than one Marker exceeds a cut-off value.

15. The method of claim 14 wherein nucleic acid amplification and detection is conducted by polymerase chain reaction (PCR).

16. The method according to claim 3 wherein mammaglobin expression is detected using oligonucleotide primers and probes selected from the group consisting of
(SEQ ID NO: 9)
AGTTGCTGATGGTCCTCATGC,
(SEQ ID NO: 10)
ATCACATTCTCCAATAAGGGGCA,
(SEQ ID NO: 11)
Fam-CCCTCTCCCAGCACTGCTACGCA-BHQ1-TT;
(SEQ ID NO: 18)
CAAACGGATGAAACTCTGAGCAATGTTGA,
(SEQ ID NO: 19)
TCTGTGAGCCAAAGGTCTTGGAGA,
(SEQ ID NO: 20)
TGTTTATGCAATTAATATATGACAGCAGTCTTTGT;
and
(SEQ ID NO: 42)
CGGATGAAACTCTGAGCAATGTTGA,
(SEQ ID NO: 43)
GAGCGAAAGGTCTTGCAGAAAGT,
(SEQ ID NO: 44)
TGTTTATGCAATTAATATATGACAGCAGTCTTTGTG.

17. The method according to claim 16 wherein the primer/probe set is
(SEQ ID NO: 9)
AGTTGCTGATGGTCCTCATGC,
(SEQ ID NO: 10)
ATCACATTCTCCAATAAGGGGCA,
(SEQ ID NO: 11)
Fam-CCCTCTCCCAGCACTGCTACGCA-BHQ1-TT.

18. The method according to claim 6 wherein CK19 expression is detected using primers and probes selected from the group consisting of:
(SEQ ID NO: 12), (SEQ ID NO: 13), (SEQ ID NO: 49);
(SEQ ID NO: 51), (SEQ ID NO: 13), (SEQ ID NO: 49);
(SEQ ID NO: 12), (SEQ ID NO: 53), (SEQ ID NO: 49);
(SEQ ID NO: 51), (SEQ ID NO: 53), (SEQ ID NO: 49);
(SEQ ID NO: 47), (SEQ ID NO: 53), (SEQ ID NO: 14);
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

19. The method according to claim 18 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

20. The method according to claim 19 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
and
(SEQ ID NO: 12), (SEQ ID NO: 13), (SEQ ID NO: 14).

21. The method according to claim 20 wherein the primers and probe are (SEQ ID NO: 12), (SEQ ID NO: 13), (SEQ ID NO: 14).

22. The method according to claim 9 wherein the primers and probe are (SEQ ID NO: 15), (SEQ ID NO: 16) and (SEQ ID NO: 17).

23. A composition comprising nucleic acid primer/probe sets selected from the group consisting of
(SEQ ID NO: 9)
AGTTGCTGATGGTCCTCATGC,
(SEQ ID NO: 10)
ATCACATTCTCCAATAAGGGGCA,
(SEQ ID NO: 11)
Fam-CCCTCTCCCAGCACTGCTACGCA-BHQ1-TT;
(SEQ ID NO: 18)
CAAACGGATGAAACTCTGAGCAATGTTGA,
(SEQ ID NO: 19)
TCTGTGAGCCAAAGGTCTTGCAGA,
(SEQ ID NO: 20)
TGTTTATGCAATTAATATATGACAGCAGTCTTTGT;
and
(SEQ ID NO: 42)
CGGATGAAACTCTGAGCAATGTTGA,
(SEQ ID NO: 43)
GAGCCAAAGGTCTTGCAGAAAGT,
and
(SEQ ID NO: 44)
TGTTTATGCAATTAATATATGACAGCAGTCTTTGTG.

24. The composition of claim 23 wherein the primer/probe set is
(SEQ ID NO: 9)
AGTTGCTGATGGTCCTCATGC,
(SEQ ID NO: 10)
ATCACATTCTCCAATAAGGGGCA,
and
(SEQ ID NO: 11)
Fam-CCCTCTCCCAGCACTGCTACGCA-BHQ1-TT.

25. A composition comprising nucleic acid primer/probe sets selected from the group consisting of
(SEQ ID NO: 12), (SEQ ID NO: 13), (SEQ ID NO: 49);
(SEQ ID NO: 51), (SEQ ID NO: 13), (SEQ ID NO: 49);
(SEQ ID NO: 12), (SEQ ID NO: 53), (SEQ ID NO: 49);
(SEQ ID NO: 51), (SEQ ID NO: 53), (SEQ ID NO: 49);
(SEQ ID NO: 47), (SEQ ID NO: 53), (SEQ ID NO: 14);
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

26. The composition according to claim 25 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

27. The composition according to claim 26 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
and
(SEQ ID NO: 12), (SEQ ID NO: 13), (SEQ ID NO: 14).

28. The composition according to claim 27 wherein the primers and probe are (SEQ ID NO: 12), (SEQ ID NO: 13), (SEQ ID NO: 14).

29. A composition comprising nucleic acid primer/probe set (SEQ ID NO: 15), (SEQ ID NO: 16) and (SEQ ID NO: 17).

30. A kit for conducting an intraoperative lymph node assay according to claim 1, comprising: nucleic acid amplification and detection reagents.

31. The kit of claim 30 wherein said reagents comprise primers having sequences for detecting the presence of a group of Markers selected from the group consisting of SEQ ID NOs: 1-8.

32. The kit of claim 31 wherein the primer/probe sets are selected from the group consisting of
(SEQ ID NO: 9)
AGTTGCTGATGGTCCTCATGC,
(SEQ ID NO: 10)
ATCACATTCTCCAATAAGGGGCA,
(SEQ ID NO: 11)
Fam-CCCTCTCCCAGCACTGCTACGCA-BHQ1-TT;
(SEQ ID NO: 18)
CAAACGGATGAAACTCTGAGCAATGTTGA,
(SEQ ID NO: 19)
TCTGTGAGCCAAAGGTCTTGCAGA,
(SEQ ID NO: 20)
TGTTTATGCAATTAATATATGACAGCAGTCTTTGT;
and
(SEQ ID NO: 42)
CGGATGAAACTCTGAGCAATGTTGA,
(SEQ ID NO: 43)
GAGCCAAAGGTCTTGCAGAAAGT,
and
(SEQ ID NO: 44).
TGTTTATGCAATTAATATATGACAGCAGTCTTTGTG.

33. The method according to claim 32 wherein the primer/probe set is
(SEQ ID NO: 9)
AGTTGCTGATGGTCCTCATGC,
(SEQ ID NO: 10)
ATCACATTCTCCAATAAGGGGCA,
(SEQ ID NO: 11)
Fam-CCCTCTCCCAGCACTGCTACGCA-BHQ1-TT.

34. The kit of claim 30 wherein the primer/probe set is selected from the group consisting of
(SEQ ID NO: 12), (SEQ ID NO: 13), (SEQ ID NO: 49);
(SEQ ID NO: 51), (SEQ ID NO: 13), (SEQ ID NO: 49);
(SEQ ID NO: 12), (SEQ ID NO: 53), (SEQ ID NO: 49);
(SEQ ID NO: 51), (SEQ ID NO: 53), (SEQ ID NO: 49);
(SEQ ID NO: 47), (SEQ ID NO: 53), (SEQ ID NO: 14);
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

35. The kit according to claim 34 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
(SEQ ID NO: 12), (SEQ ID NO: 13), (SEQ ID NO: 14);
(SEQ ID NO: 51), (SEQ ID NO: 13), (SEQ ID NO: 14);
and
(SEQ ID NO: 52), (SEQ ID NO: 13), (SEQ ID NO: 14).

36. The kit according to claim 35 wherein the primers and probes are selected from the group consisting of
(SEQ ID NO: 12), (SEQ ID NO: 53), (SEQ ID NO: 14);
(SEQ ID NO: 51), (SEQ ID NO: 53), (SEQ ID NO: 14);
(SEQ ID NO: 52), (SEQ ID NO: 53), (SEQ ID NO: 14);
and
(SEQ ID NO: 12), (SEQ ID NO: 13), (SEQ ID NO: 14).

37. The kit according to claim 30 wherein the primers and probe are (SEQ ID NO: 12), (SEQ ID NO: 13), (SEQ ID NO: 14).

38. The kit according to claim 30 wherein the primers and probe are (SEQ ID NO: 15), (SEQ ID NO: 16) and (SEQ ID NO: 17).

39. The kit of claim 30 comprising RT-PCR reagents.
